(19) United States
(12) Patent Application Publication
(10) Pub. No.: US 2025/0381383 A1
(43) Pub. Date: Dec. 18, 2025

(54) Decorative Nebulizer Cap Device

(71) Applicant: Jennifer Riggs, Las Vegas, NV (US)

(72) Inventor: Jennifer Riggs, Las Vegas, NV (US)

(21) Appl. No.: 19/218,721

(22) Filed: May 27, 2025

Related U.S. Application Data
(60) Provisional application No. 63/660,756, filed on Jun. 17, 2024.

(51) Int. Cl. A61M 39/20

(52) U.S. Cl. CPC A61M 39/20; A61M 2205/59

(57) ABSTRACT

A decorative nebulizer cap device is provided. The device is comprised of a decorative nebulizer cap device configured to maintain the hygiene of a nebulizer mouthpiece between treatment sessions by enclosing the mouthpiece opening to block environmental contaminants such as dust and bacteria. The device comprises a cap shaped to fit the mouthpiece. In one embodiment, the cap includes an antimicrobial cloth cover with an elastic edge to ensure a secure fit. A tether may secure the cap to the mouthpiece to prevent loss when removed in one embodiment. Additionally, the cap supports an interchangeable decorative attachment, affixed via various fasteners such as magnets, clips, or threaded interfaces, allowing users to personalize the device with aesthetic elements like figures, logos, or designs.

### CROSS-REFERENCE TO RELATED APPLICATION

The present application claims priority to, and the benefit of, U.S. Provisional Application No. 63/660,756, which was filed on Jun. 17, 2024, and is incorporated herein by reference in its entirety.

### FIELD OF THE INVENTION

The present invention relates generally to the field of nebulizers. More specifically, the present invention relates to a decorative cap for a nebulizer mouthpiece. Accordingly, the present disclosure makes specific reference thereto. Nonetheless, it is to be appreciated that aspects of the present invention are also equally applicable to other like applications, devices, and methods of manufacture.

### BACKGROUND

The use of nebulizers is a common and medically necessary practice for individuals managing chronic respiratory conditions such as asthma, chronic obstructive pulmonary disease (COPD), and other pulmonary disorders. These devices administer aerosolized medication through a mouthpiece, enabling effective delivery directly to the lungs. However, the nebulizer mouthpiece is often left exposed to the surrounding environment between treatment sessions. This exposure presents a significant hygiene issue, as the mouthpiece may accumulate airborne particulates, dust, bacteria, and other environmental contaminants. Over time, this contamination can compromise the sterility of the device and increase the risk of infection for users. Despite clinical recommendations to clean or replace the mouthpiece cap regularly, many users neglect this practice due to inconvenience, lack of awareness, or physical limitations. The repeated use of an unsanitary mouthpiece can introduce pathogens into the respiratory tract, exacerbating medical conditions and potentially leading to secondary infections.

Therefore, there exists a long-felt need in the art for the decorative nebulizer cap device that prevents environmental contaminants from entering the nebulizer mouthpiece between uses. There also exists a long-felt need in the art for the decorative nebulizer cap device that accommodates a variety of mouthpiece shapes and sizes through a universal and flexible design. Moreover, there exists a long-felt need in the art for the decorative nebulizer cap device that encourages consistent use through customizable and aesthetically engaging features.

The subject matter disclosed and claimed herein, in one embodiment thereof, comprises a decorative nebulizer cap device. The device is comprised of a decorative nebulizer cap device configured to maintain the hygiene of a nebulizer mouthpiece between treatment sessions by enclosing the mouthpiece opening to block environmental contaminants such as dust and bacteria. The device comprises a cap shaped to fit the mouthpiece. In one embodiment, the cap includes an antimicrobial cloth cover with an elastic edge to ensure a secure fit. A tether may secure the cap to the mouthpiece to prevent loss when removed in one embodiment. Additionally, the cap supports an interchangeable decorative attachment, affixed via various fasteners such as magnets, clips, or threaded interfaces, allowing users to personalize the device with aesthetic elements like figures, logos, or designs.

In this manner, the decorative nebulizer cap device of the present invention accomplishes all the foregoing objectives and provides a protective device that securely covers a nebulizer mouthpiece, thereby reducing exposure to contaminants such as dust, bacteria, and other irritants. The inclusion of an antimicrobial cloth cover with an elastic edge ensures compatibility with various mouthpiece designs while maintaining a secure fit. Additionally, the decorative attachment mechanism enables personalization through a range of visual designs, enhancing user engagement and promoting consistent hygiene practices. The tethering system further prevents misplacement. Collectively, these features ensure that the decorative nebulizer cap device effectively improves safety, usability, and user compliance in respiratory therapy contexts.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a decorative nebulizer cap device. The decorative nebulizer cap device is designed to maintain the hygiene of a nebulizer mouthpiece by covering the mouthpiece opening between treatment sessions. This coverage prevents exposure to airborne particulates, dust, bacteria, and other contaminants, thereby reducing the risk of user infection during repeated use.

The device is comprised of a cap that encloses the nebulizer mouthpiece. The cap may be formed in various geometric shapes to accommodate different mouthpiece configurations. Depending on the embodiment, the cap may be configured as a snap-fit, press-fit, threaded, or friction-fit cap to ensure a secure connection with the mouthpiece.

In one embodiment, the cap is comprised of an antimicrobial cloth cover formed from treated fabric or synthetic textiles. The cloth cover includes an elastic edge that stretches over the mouthpiece to form a tight seal. The antimicrobial treatment includes agents such as silver ions or copper-based compounds to inhibit microbial growth. The cap may also be comprised of a tether for securing the cap to the nebulizer mouthpiece.

The device further includes an interchangeable decorative attachment that can be removably secured to the cap. This attachment is connected using reciprocating fasteners that allow for easy replacement of the decorative attachment without interfering with the primary function of the cap. The decorative attachment may comprise various two-dimensional or three-dimensional forms such as animal figures, characters, symbols, letters, logos, or artistic designs. The customization features are intended to improve user engagement and treatment compliance, especially among pediatric users.

A method of using one embodiment of the device is comprised of providing a cap and decorative attachment, placing the cap over the nebulizer mouthpiece to cover the opening, securing the tether around the mouthpiece, and affixing the decorative attachment to the cap using a selected reciprocating fastener to achieve personalization.

Accordingly, the decorative nebulizer cap device of the present invention is particularly advantageous as it provides a protective device that securely covers a nebulizer mouthpiece, thereby reducing exposure to contaminants such as dust, bacteria, and other irritants. The inclusion of an antimicrobial cloth cover with an elastic edge ensures compatibility with various mouthpiece designs while maintaining a secure fit. Additionally, the decorative attachment mechanism enables personalization through a range of visual designs, enhancing user engagement and promoting consistent hygiene practices. The tethering system further prevents misplacement. Collectively, these features ensure that the decorative nebulizer cap device effectively improves safety, usability, and user compliance in respiratory therapy contexts.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a decorative nebulizer cap device of the present invention while attached to a nebulizer in accordance with the disclosed architecture;

FIG. 2 illustrates a perspective view of a cap and a decorative attachment of one potential embodiment of a decorative nebulizer cap device of the present invention while unattached in accordance with the disclosed architecture; and

FIG. 3 illustrates a flowchart of a method of using one potential embodiment of a decorative nebulizer cap device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a decorative nebulizer cap device that prevents environmental contaminants from entering the nebulizer mouthpiece between uses. There also exists a long-felt need in the art for the decorative nebulizer cap device that accommodates a variety of mouthpiece shapes and sizes through a universal and flexible design. Moreover, there exists a long-felt need in the art for the decorative nebulizer cap device that encourages consistent use through customizable and aesthetically engaging features.

The present invention, in one exemplary embodiment, is comprised of a decorative nebulizer cap device. The decorative nebulizer cap device is configured to preserve the hygiene of a nebulizer mouthpiece by covering the mouthpiece opening between treatment sessions. This protective coverage minimizes exposure to airborne particles, dust, bacteria, and other contaminants, thereby lowering the risk of infection during repeated use.

The device is comprised of a cap that encloses the nebulizer mouthpiece. The cap may be formed in a range of geometric shapes to accommodate varying mouthpiece designs. Depending on the embodiment, the cap may be configured as a snap-fit, press-fit, threaded, or friction-fit cap to ensure a secure attachment to the mouthpiece.

In one embodiment, the cap is comprised of an antimicrobial cloth cover made from treated fabric or synthetic textiles. The cloth cover features an elastic edge designed to stretch over the mouthpiece and create a sealed fit. The antimicrobial treatment utilizes agents such as silver ions or copper-based compounds to prevent microbial growth. The cap may further be comprised of a tether configured to secure the cap to the nebulizer mouthpiece.

The device also includes an interchangeable decorative attachment that can be removably affixed to the cap. The attachment is connected using reciprocating fasteners that enable convenient replacement without affecting the primary function of the cap. The decorative attachment may take the form of various two-dimensional or three-dimensional figures, including animals, characters, symbols, letters, logos, or artistic shapes. These customization features are intended to enhance user interaction and support treatment adherence, particularly for pediatric users.

One method of using an embodiment of the device is comprised of providing a cap and a decorative attachment, placing the cap over the nebulizer mouthpiece to cover the opening, securing the tether around the mouthpiece, and attaching the decorative component to the cap using a selected reciprocating fastener for personalization.

As a result, the decorative nebulizer cap device offers significant advantages by serving as a protective element that effectively covers the nebulizer mouthpiece and minimizes exposure to contaminants such as dust, bacteria, and irritants. The antimicrobial cloth cover with an elastic edge supports compatibility with various mouthpiece designs while maintaining a secure fit. The decorative attachment system enables user-specific customization through diverse visual elements, enhancing engagement and promoting regular hygiene practices. The tethering feature further reduces the risk of loss. Collectively, these attributes contribute to improved safety, functionality, and user compliance in respiratory therapy applications.

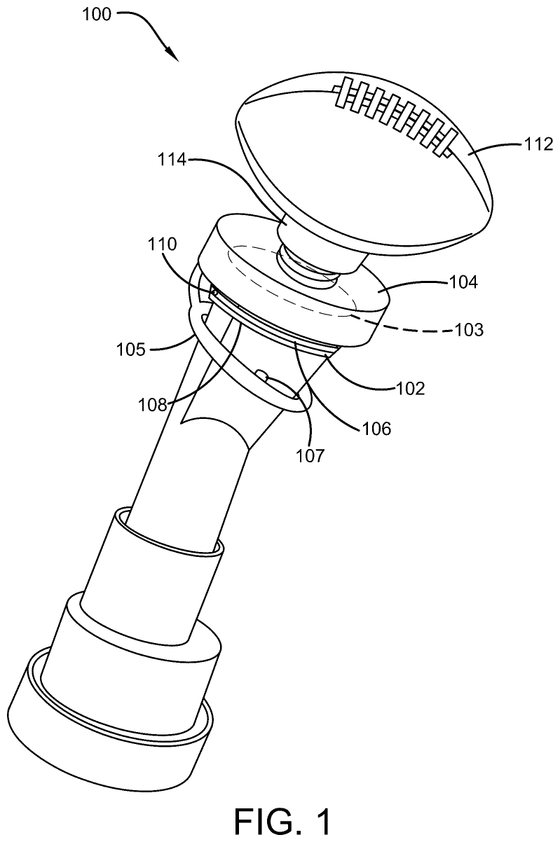

Referring initially to the drawings, FIG. 1 illustrates a perspective view of one potential embodiment of a decorative nebulizer cap device 100 of the present invention while attached to a nebulizer in accordance with the disclosed architecture. The decorative nebulizer cap device 100 is configured to maintain the hygiene of a nebulizer mouthpiece 102 between treatment sessions for individuals managing respiratory conditions such as asthma, chronic obstructive pulmonary disease (COPD), and other pulmonary illnesses. The device 100 functions by covering the opening 103 of the mouthpiece 102, thereby preventing exposure to environmental contaminants such as airborne particulates, dust, bacteria, and other potential sources of infection that may accumulate between uses. By shielding the mouthpiece 102, the device 100 reduces the likelihood that a user will be exposed to contaminants when placing the mouthpiece 102 back into the mouth during subsequent treatment sessions.

The device 100 is comprised of a cap 104 that is shaped and dimensioned to enclose the mouthpiece 102, as seen in FIG. 1. The cap 104 may take a variety of geometric forms, including cylindrical, conical, cuboidal, hemispherical, or hybrid shapes, depending on the specific configuration of the mouthpiece 102. In one embodiment, the cap 104 is a snap-fit cap that engages the mouthpiece 102 through elastic or resilient deformation to form a secure connection. In alternative embodiments, the cap 104 may be configured as a press-fit cap, a threaded cap, or a friction-fit cap, with each design offering different performance characteristics based on manufacturing needs, mouthpiece geometry, and user preferences.

In one embodiment, the cap 104 is comprised of an antimicrobial cloth cover 106, as seen in FIG. 1. The antimicrobial cloth cover 106 is formed from a fabric or synthetic textile treated with antimicrobial agents 110 such as silver ions, copper-based compounds, or other known antimicrobial additives. These agents 110 are applied to inhibit microbial growth on the surface of the cap 104. The cloth cover 106 includes an elastic edge 108 that allows it to stretch over and conform to a range of mouthpiece 102 shapes and sizes, ensuring a tight and effective seal. The elastic edge 108 may be fabricated from spandex-infused fibers, woven elastic threads, or integrated elastic bands.

In other embodiments, the cap 104 may be constructed from rigid or semi-rigid materials such as silicone, thermoplastic elastomers (TPE), polypropylene, or acrylonitrile butadiene styrene (ABS). These materials may also incorporate antimicrobial fillers that function similarly to the treated fabrics. In one embodiment, the cap 104 is further comprised of a tether 105 that is configured to attach around the mouthpiece 102, as seen in FIG. 1. The tether 105 may be a looped tether formed from elastic cord, woven strap, or flexible plastic tubing, allowing it to maintain continuous attachment to the mouthpiece 102. In one configuration, the tether 105 is fixed at one end to the cap 104 and forms a closed loop that can be slipped over the base of the mouthpiece 102. In another configuration, the tether 105 may include a fastener 107 such as a snap button, hook-and-loop strip, or adjustable slider, enabling the tether 105 to wrap around the mouthpiece 102 and be secured in place. The tether 105 ensures that the cap 104 remains connected to the nebulizer assembly even when removed from the mouthpiece 102, thereby preventing accidental loss or misplacement.

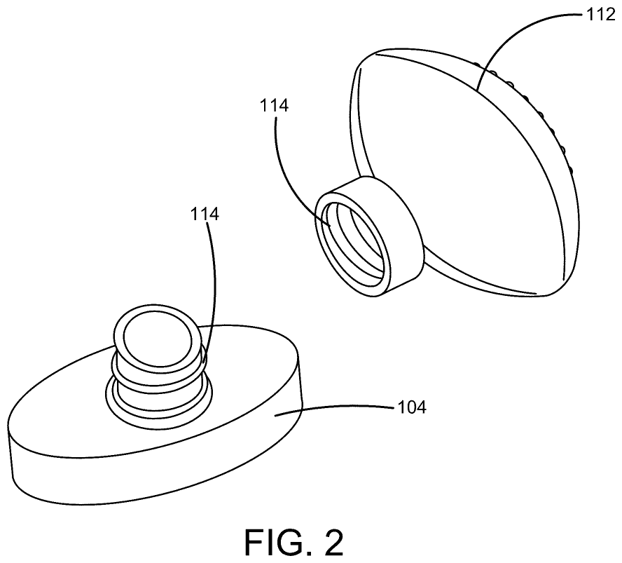

The decorative nebulizer cap device 100 also includes an interchangeable decorative attachment 112 that can be removably secured to the cap 104, as seen in FIG. 2. The decorative attachment 112 is connected using reciprocating fasteners 114, which may include threaded interfaces, hook-and-loop strips, embedded magnets, snap-fit clips, press-fit connections, or bayonet-style latches. The specific fastening method may be selected based on the intended user demographic, aesthetic goals, or manufacturing constraints, and allows the decorative attachment 112 to be easily removed and replaced without impacting the primary function of the cap 104.

The decorative attachment 112 may take the form of a wide variety of two-dimensional or three-dimensional elements, including animal figures, characters, symbols, letters, numbers, logos, or artistic designs. These components may be produced from polymers, foam materials, composite resins, soft fabrics, or lightweight metals, allowing for variation in texture and appearance. The customization capability offered by the decorative attachment 112 serves both aesthetic and behavioral purposes, enhancing user engagement with the nebulizer device 100 and potentially improving compliance, particularly among pediatric users who may otherwise resist routine medical treatments.

In summary, the decorative nebulizer cap device 100 improves hygienic conditions for nebulizer users by sealing the mouthpiece 102 against environmental exposure between treatment sessions. At the same time, the customizable decorative attachment 112 enhances the user experience by offering personalization options that can be tailored to different preferences, ages, or branding requirements. The inclusion of antimicrobial materials, a tether 105 for cap retention, and adaptable fastener mechanisms ensures that the device 100 remains functional, safe, and user-friendly across a wide range of applications.

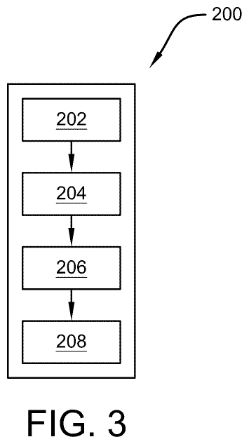

The decorative nebulizer cap device 100 is also comprised of a method of use 200, as seen in FIG. 3. First, a device 100 is provided that is comprised of a cap 104 and a decorative attachment 112 [Step 202]. Then, the cap 104 is placed over the nebulizer mouthpiece 102 such that the cap 104 covers the opening 103 of the mouthpiece 102 to create a protective barrier [Step 204]. In one embodiment, a tether 105 is secured around the mouthpiece 102, either by sliding a looped tether over the base of the mouthpiece 102 or by wrapping and fastening the tether 105 using a fastener 107 such as a snap, slider, or hook-and-loop segment [Step 206]. Finally, a decorative attachment 112 is affixed to the cap 104 using the selected reciprocating fastener 114 to personalize the appearance of the device 100 according to user preference [Step 208].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein, “decorative nebulizer cap device” and “device” are interchangeable and refer to the decorative nebulizer cap device 100 of the present invention.

Notwithstanding the foregoing, the decorative nebulizer cap device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the decorative nebulizer cap device 100, as shown in the FIGS. are for illustrative purposes only, and that many other sizes and shapes of the decorative nebulizer cap device 100 are well within the scope of the present disclosure. Although the dimensions of the decorative nebulizer cap device 100 are important design parameters for user convenience, the decorative nebulizer cap device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A decorative nebulizer cap device comprising:
a cap shaped and dimensioned to enclose a mouthpiece of a nebulizer;
a decorative attachment removably secured to the cap via a fastener; and
an antimicrobial cloth cover comprised of an elastic edge configured to stretch over and conform to the mouthpiece.

2. The decorative nebulizer cap device of claim 1, wherein the fastener is comprised of a magnet.

3. The decorative nebulizer cap device of claim 1, wherein the fastener is comprised of a snap-fit clip.

4. The decorative nebulizer cap device of claim 1, wherein the fastener is comprised of a press-fit connection.

5. The decorative nebulizer cap device of claim 1, wherein the antimicrobial cloth cover is comprised of an antimicrobial agent.

6. The decorative nebulizer cap device of claim 5, wherein the anti-microbial agent is comprised of a silver ion or a copper-based compound.

7. The decorative nebulizer cap device of claim 1, wherein the decorative attachment is comprised of an animal figure, a character, a symbol, a letter, a number, a logo, or an artistic design.

8. A decorative nebulizer cap device comprising:
a cap shaped and dimensioned to enclose a mouthpiece of a nebulizer, the cap comprised of a first fastener;
a tether connected to the cap and configured to attach around the mouthpiece;
a decorative attachment removably secured to the cap via a second fastener that engages the first fastener; and
an antimicrobial cloth cover comprised of an elastic edge configured to stretch over and conform to the mouthpiece.

9. The decorative nebulizer cap device of claim 8, wherein the tether is comprised of a third fastener.

10. The decorative nebulizer cap device of claim 9 wherein the third fastener is comprised of a snap button or a hook and loop fastener.

11. The decorative nebulizer cap device of claim 8, wherein the tether is comprised of an elastic cord.

12. The decorative nebulizer cap device of claim 8, wherein the first fastener and the second fastener are comprised of a pair of threads, a pair of hook and loop fasteners, or a pair of magnet fasteners.

13. The decorative nebulizer cap device of claim 8, wherein the antimicrobial cloth cover is comprised of an antimicrobial agent.

14. The decorative nebulizer cap device of claim 8, wherein the decorative attachment is comprised of an animal figure, a character, a symbol, a letter, a number, a logo, or an artistic design.

15. The decorative nebulizer cap device of claim 8, wherein the antimicrobial cloth cover is comprised of an antimicrobial agent.

16. The decorative nebulizer cap device of claim 15, wherein the anti-microbial agent is comprised of a silver ion or a copper-based compound.

17. The decorative nebulizer cap device of claim 8, wherein the tether is fixedly attached to the cap.

18. A method of using a decorative nebulizer cap device, the method comprising the following steps:
providing a decorative nebulizer cap device comprised of a cap and a decorative attachment attached to the cap;
placing the cap over a mouthpiece of a nebulizer such that the cap covers an opening of the mouthpiece to create a protective barrier; and
attaching the decorative attachment to the cap.

19. The method of using a decorative nebulizer cap device of claim 18, wherein the decorative attachment attaches to the cap via a fastener.

20. The method of using a decorative nebulizer cap device of claim 18 further comprised of attaching a tether around the mouthpiece.
